(19) United States
(12) Patent Application Publication
(10) Pub. No.: US 2022/0117903 A1
(43) Pub. Date: Apr. 21, 2022

(54) DRUG DELIVERY SYSTEM AND METHOD

(71) Applicants: SEOUL NATIONAL UNIVERSITY R&DB FOUNDATION, Seoul (KR); INSTITUTE FOR BASIC SCIENCE, Daejeon (KR)

(72) Inventors: Taeghwan HYEON, Seoul (KR); Soohong LEE, Seoul (KR); Jonghoon KIM, Seoul (KR); Okkyu PARK, Seoul (KR); Nohyun LEE, Seoul (KR)

(21) Appl. No.: 17/422,414

(22) PCT Filed: Jan. 16, 2020
(86) PCT No.: PCT/KR2020/000783

§ 371 Date: Jul. 12, 2021

(30) Foreign Application Priority Data
Jan. 16, 2019 (CN) 10-2019-0005578

(51) Int. Cl. A61K 9/51; A61P 35/00; A61K 47/68; A61K 47/69

(52) U.S. Cl. CPC A61K 9/5123; A61P 35/00; A61K 9/5146; A61K 47/6901; A61K 9/5115; A61K 47/6849

(57) ABSTRACT

A drug delivery system and a drug delivery method are provided. The drug delivery system comprises a nanoparticle to which a first functional group is bound and drug is loaded, an antibody to which a second functional group to react with the first functional group is bound, and a carrier cell comprising an antigen protein to bind to the antibody. The drug delivery method comprises injecting a nanoparticle to which a first functional group is bound and drug is loaded, and an antibody to which a second functional group to react with the first functional group is bound into a living body, and binding the antibody to an antigen protein of a carrier cell present in the living body, and binding the nanoparticle to the antibody by reaction of the first functional group and the second functional group.

### TECHNICAL FIELD

The present invention relates to a drug delivery system and a drug delivery method.

### BACKGROUND ART

Nanoparticles have been used extensively to deliver therapeutic drugs to tumor tissues through the targeted interaction of leaky blood vessels or tumor-specific ligands. However, because the drug-loaded nanoparticles have limited penetration into the tumor tissue, the drug cannot be effectively delivered to the tumor due to the heterogeneous distribution of the nanoparticles.

### DISCLOSURE

### Technical Problem

In order to solve the above mentioned problems, the present invention provides a drug delivery system capable of effectively delivering a drug in a living body.

The present invention provides a drug delivery method capable of effectively delivering a drug in a living body.

The other objects of the present invention will be clearly understood by reference to the following detailed description and the accompanying drawings.

### Technical Solution

A drug delivery system according to the embodiments of the present invention comprises a nanoparticle to which a first functional group is bound and drug is loaded, an antibody to which a second functional group to react with the first functional group is bound, and a carrier cell comprising an antigen protein to bind to the antibody.

A drug delivery method according to the embodiments of the present invention comprises injecting a nanoparticle to which a first functional group is bound and drug is loaded, and an antibody to which a second functional group to react with the first functional group is bound into a living body, and binding the antibody to an antigen protein of a carrier cell present in the living body, and binding the nanoparticle to the antibody by reaction of the first functional group and the second functional group.

### Advantageous Effects

According to the embodiments of the present invention, it is possible to effectively deliver a drug to a target site in vivo, such as a tumor. Drug-loaded nanoparticles can penetrate deep into the tumor, improving the efficacy of tumor treatment. It does not require in vitro manipulation of cells and can be applied to various types of cells and nanovehicles.

### DESCRIPTION OF DRAWINGS

FIG. 1 is a view for explaining a drug delivery system and a drug delivery method according to an embodiment of the present invention.

FIG. 2 shows mesoporous silica nanoparticles (MSN-Tz) functionalized with tetrazine.

FIG. 3 shows a TEM image of the MSN-Tz of FIG. 2.

FIG. 4 shows the hydrodynamic diameter of MSN-Tz according to the concentration of tetrazine (Tz).

FIG. 5 shows the accumulated release profile of doxorubicin from MSN-Tz loaded with doxorubicin.

FIGS. 6 and 7 are views for explaining the effect of Tz and TCO on the binding of MSN and anti-CD11b antibody.

FIG. 8 shows the change in correlation between MSN-Tz and anti-CD11b-TCO (anti-CD11b antibody functionalized with TCO) depending on time.

FIG. 9 shows the conjugation efficiency of MSN-Tz and anti-CD11b-TCO depending on time.

FIG. 10 shows the evaluation results of the in vitro cytotoxicity of MSN-Tz loaded with doxorubicin using the MTS assay.

FIG. 11 shows the experimental results of trypan blue quenching for MSN-Tz.

FIG. 12 shows the evaluation results of the migration capability of cells to which MSN-Tz and anti-CD11b-TCO are bound.

FIG. 13 shows confocal microscopy images showing the targeting of MSN-Tz to anti-CD11b-TCO at the surface of bone marrow cells.

FIGS. 14 to 21 are views for explaining the delivery of MSN-Tz to a 4T1 tumor in a living body using a drug delivery method according to an embodiment of the present invention.

FIGS. 22 to 25 are views for explaining the therapeutic efficacy of MSN-Tz improved by the drug delivery method according to an embodiment of the present invention.

### BEST MODE

Hereinafter, a detailed description will be given of the present invention with reference to the following embodiments. The purposes, features, and advantages of the present invention will be easily understood through the following embodiments. The present invention is not limited to such embodiments, but may be modified in other forms. The embodiments to be described below are nothing but the ones provided to bring the disclosure of the present invention to perfection and assist those skilled in the art to completely understand the present invention. Therefore, the following embodiments are not to be construed as limiting the present invention.

Terms like ‘first’, ‘second’, etc., may be used to indicate various components, but the components should not be restricted by the terms. These terms are only used to distinguish one component from another component.

The terminology used herein is for the purpose of describing particular embodiments only and is not intended to limit the invention. It is to be understood that the singular forms “a,” “an,” and “the” include plural references unless the context clearly dictates otherwise. It will be further understood that the terms “comprises” or “has,” when used in this specification, specify the presence of stated features, integers, steps, operations, elements, and/or components, but do not preclude the presence or addition of one or more other features, integers, steps, operations, elements, components, and/or groups thereof.

A drug delivery system according to the embodiments of the present invention comprises a nanoparticle to which a first functional group is bound and drug is loaded, an antibody to which a second functional group to react with the first functional group is bound, and a carrier cell comprising an antigen protein to bind to the antibody.

A drug delivery method according to the embodiments of the present invention comprises injecting a nanoparticle to which a first functional group is bound and drug is loaded, and an antibody to which a second functional group to react with the first functional group is bound into a living body, and binding the antibody to an antigen protein of a carrier cell present in the living body, and binding the nanoparticle to the antibody by reaction of the first functional group and the second functional group.

The first functional group and the second functional group may be bonded by a click reaction. The first functional group may comprise tetrazine and the second functional group may comprise trans-cyclooctene.

The nanoparticle may comprise polyethylene glycol disposed on the surface, and the first functional group may bind to the polyethylene glycol. The nanoparticle may comprise mesoporous silica nanoparticle. The antibody may comprise an anti-CD11b antibody. The carrier cell may comprise a myeloid-derived suppressor cell. The antigen protein may comprise CD11b.

The nanoparticle may penetrate a tumor in the living body by the carrier cell.

### EMBODIMENTS

[Preparation Example of Amine-Functionalized Mesoporous Silica Nanoparticles (MSNs)]

To prepare fluorescently labeled mesoporous silica nanoparticles (MSNs), fluorescent dye-silane derivatives are first formed. The fluorescent dye-silane derivatives may be formed by conjugating (3-aminopropyl)triethoxysilane and fluorescent dyes. The fluorescent dyes may include rhodamine B isothiocyanate, cyanine 5 NHS ester (Cy5) and cyanine 5.5 NHS ester (Cy5.5). Each dye is dissolved in ethanol at 3 mM concentration with (3-aminopropyl)triethoxysilane at 15 mM. This mixture is shaken at room temperature. 2 g of hexadecyl trimethyl ammonium chloride (25% cetyltrimethylammonium chloride solution, 8 ml) and 80 mg of triethanolamine are dissolved in 20 ml of distilled water. The mixture is heated at 95° C. for 1 hour and then 1.5 ml of tetraethyl orthosilicate is added. Then, the dye-silane derivatives are added. After 50 min, the reaction is stopped and the products are collected by centrifugation and redispersed with ethanol several times. To extract residual surfactant in MSNs, the resulting MSNs are stirred in ammonium nitrate (60 mg/ml in methanol) for 1 hour, and the same extraction process is repeated twice. Amine-functionalized MSNs are prepared by adding 150 μg of (3-aminopropyl)triethoxysilane and reacting at 80° C. for 3 hours. The amine-functionalized MSNs are dispersed in ethanol at a concentration of 20 mg/ml.

[Preparation Example of MSN-Tz (MSN Functionalized with Tz)]

Fmoc-PEGSK-SCM (Fluorenylmethyloxycarbonyl-poly(ethylene oxide) 5K-succinimidyl NHS acid ester) and mPEG2K-SCM (methoxy-poly(ethylene oxide) 2K-succinimidyl NHS acid ester) are prepared. PEG derivatives are dissolved in dimethylformamide (DMF) at 100 mg/ml. Fmoc-PEG5K-SCM (5 mg) is added to the suspension of amine-functionalized MSNs (20 mg) at 25° C. and stirred for 6 hours to PEGylate MSNs. mPEG2K-SCM is added to this mixture and stirred for 6 hours. The products are purified by centrifugation (15000 rpm, 20 min) and redispersed in DMF (5 ml). 1 ml of piperidine is added to the products and stirred at 25° C. for 1 hour to remove Fmoc protecting group. After several purification processes by centrifugation, methyltetrazine-PEG4-NHS ester (2.2 mg) is added to the mixture at 25° C. for 3 hours to form MSNs-Tz.

The mixture is washed with deionized water and redispersed in 5% glucose solution. To determine the number of Tz moiety per one MSN, 1 ml of MSNs-Tz (4 mg/ml) is reacted with 200 μl of Cy5-TCO (1 mg/ml) at 25° C. for 1 hour. After several purification processes by centrifugation, the absorption of nanoparticles is characterized by UV-Vis absorption spectroscopy.

[Preparation Example of Anti-CD11b-TCO (Anti-CD11b Antibody Functionalized with TCO)]

Monoclonal antibody (anti-CD11b) is dissolved in 0.1 M NaHCO₃ buffer (pH 8.5) to a final concentration of 2 mg/ml. This solution is incubated with 3 equivalents of fluorescent succinimidyl ester at 25° C. for 3 hours. The antibody is purified by centrifuge filtration and stored in phosphate buffered saline (PBS). The concentration of the antibody and the number of fluorescence dyes per antibody are confirmed by spectrophotometric analysis. The ratio of antibody and fluorescence dye is tuned to one. To label the fluorescent antibody with trans-cyclooctene (TCO), the antibody is dissolved in 0.1M NaHCO₃ buffer solution and incubated with TCO-PEG4-NHS (9 equivalents) at 4° C. for 13 hours. Amine-reactive TCO-PEG4-NHS is dissolved in anhydrous DMF to make a stock solution (5 mg/ml). After the reaction, the antibody is purified by centrifuge filtration using PBS and stored at 4° C.

To determine the number of TCO moiety per antibody, anti-CD11b-TCO is diluted with PBS (1 mg/ml) and reacted with Cy3-Tz (2 equivalents) dissolved in DMF in advance (1 mg/ml). After reaction at room temperature for 1 hour, the produced antibody is purified by centrifuge filtration using PBS. The absorption of the labeled antibody is measured with UV-Vis absorption spectroscopy.

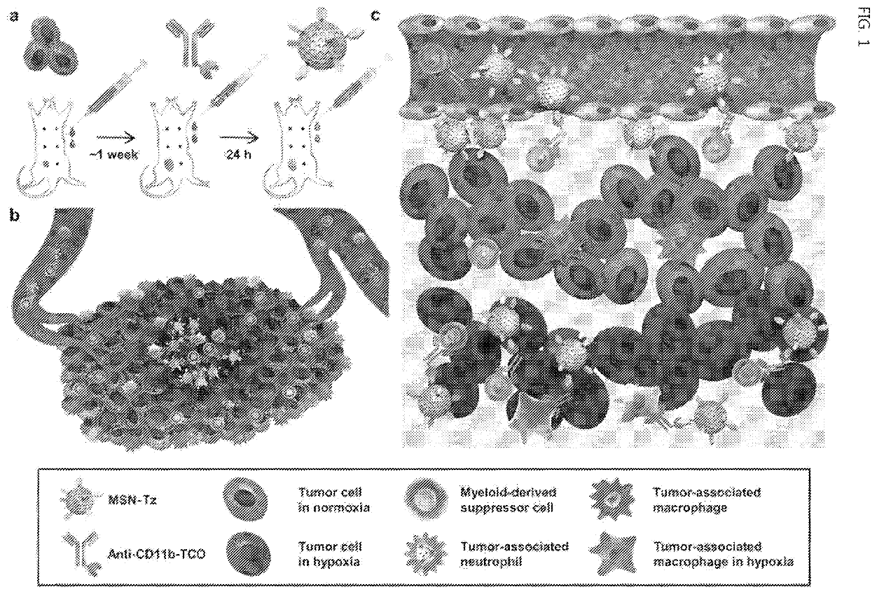

FIG. 1 is a view for explaining a drug delivery system and a drug delivery method according to an embodiment of the present invention.

Referring to FIG. 1, myeloid-derived suppressor cells (MDSCs) can serve as carrier cells because the MDSCs are typically rapidly recruited at the early stage in various tumor types to protect tumor cells from immune destruction by inhibiting T cell function. Moreover, MDSCs can infiltrate deep inside the tumor, far from blood vessels where hypoxia is frequently developed, and can differentiate into tumor-associated macrophages (TAMs). Therefore, autologous MDSCs can be used as a transporter to deliver doxorubicin (DOX)-loaded nanoparticles inside tumors that nanoparticles cannot access due to the enhanced permeability and retention (EPR) effect, and the therapeutic efficacy of doxorubicin (anticancer drug) can be increased. These MDSCs decorated with nanoparticles can serve as local drug reservoirs that release doxorubicin to adjacent tumor cells.

However, high binding selectivity and specificity are required to achieve in vivo conjugation of nanoparticles to MDSCs in circulation and in the tumor microenvironment. According to an embodiment of the present invention, click chemistry is used for surface functionalization to enhance the selectivity and specificity of nanoparticles. A rapid, selective, and high-yielding click reaction is used in living systems. Chemical combinations including azide-alkyne, thiol-ene and Diels-Alder can be used for biocompatible click reactions. In particular, the inverse Diels-Alder cycloaddition reaction between 1,2,4,5-tetrazine (Tz) and trans-cyclooctene (TCO) proceeds faster than other click reactions.

For rapid in vivo catalytic-free reaction, Tz/TCO cycloaddition can be used to selectively target drug-loaded nanoparticles to MDSCs in the circulation and tumor microenvironment.

Primary administration of TCO-functionalized CD11b antibody (anti-CD11b-TCO) allows Tz-functionalized mesoporous silica nanoparticles (MSNs-Tz) to bind to CD11b⁺ bone marrow cells. Labeled CD11b⁺ cells are unaffected by doxorubicin molecules loaded in MSNs and maintain their mobility toward 4T1 cancer cells in the body.

Real-time intravital imaging of 4T1 tumor-bearing mice shows that CD11b⁺ cells targeted with MSNs-Tz are highly motile and migrate in tumor vasculatures. CD11b⁺ cell-mediated delivery shows a uniform distribution and deep tumor penetration of MSNs-Tz.

MSNs-Tz delivered inside the tumor according to the above drug delivery system and method show much deeper penetration up to 2.5 mm compared to nanoparticles delivered by EPR effect. Doxorubicin delivery rapidly reduces tumors without systemic toxicity.

MDSCs can have a monocytic (CD11b⁺ Ly6⁺ Ly6G⁻) or polymorphonuclear morphology (CD11b⁺ Ly6C^{low} Ly6G+) with different levels of surface proteins. To identify antibodies that most efficiently target the surface of MDSCs in tumors, anti-CD11b antibodies, anti-Ly6G antibodies, and anti-Ly6C antibodies are functionalized with near infraraed (NIR) fluorescent dye (Alexa Fluor 680) and intravenously injected into mice bearing 4T1 breast tumor. Among them, the anti-CD11b antibodies show the greatest accumulation in whole tumor regions after 24 hours. Ex vivo immunohistochemical staining of the tumor slice shows that CD11b⁺ cells are uniformly distributed in both the periphery and interior of the tumor, suggesting that CD11b integrin on the surface of MDSCs is a good target for the 4T1 breast tumor microenvironment.

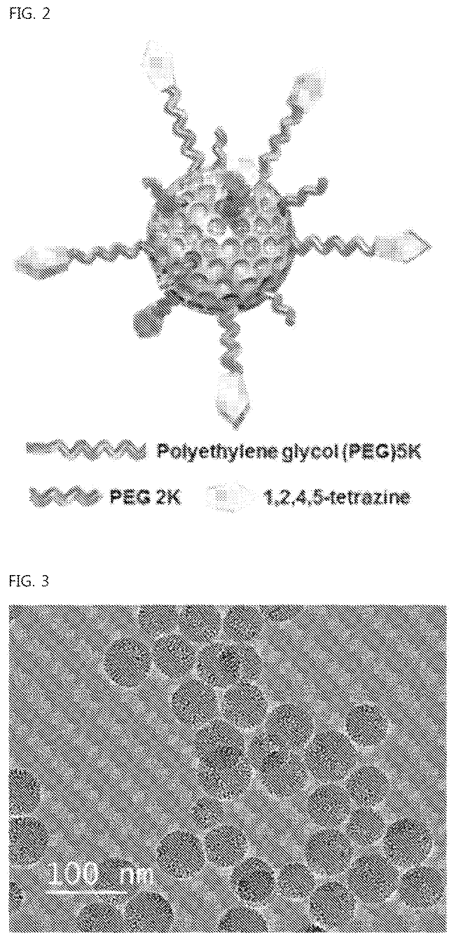

FIGS. 2 to 9 are views for explaining a manufacturing method and characteristics of the MSN-Tz.

Referring to FIG. 2, fluorescent dyes for imaging are encapsulated within the silica matrix of MSNs and doxorubicin is loaded inside the mesopores. To prevent nanoparticle uptake by the mononuclear phagocyte system, the MSN surface is functionalized with polyethylene glycol (PEG), and Tz molecules are attached to the terminal end of the PEG to allow quick access to the TCO-functionalized antibodies.

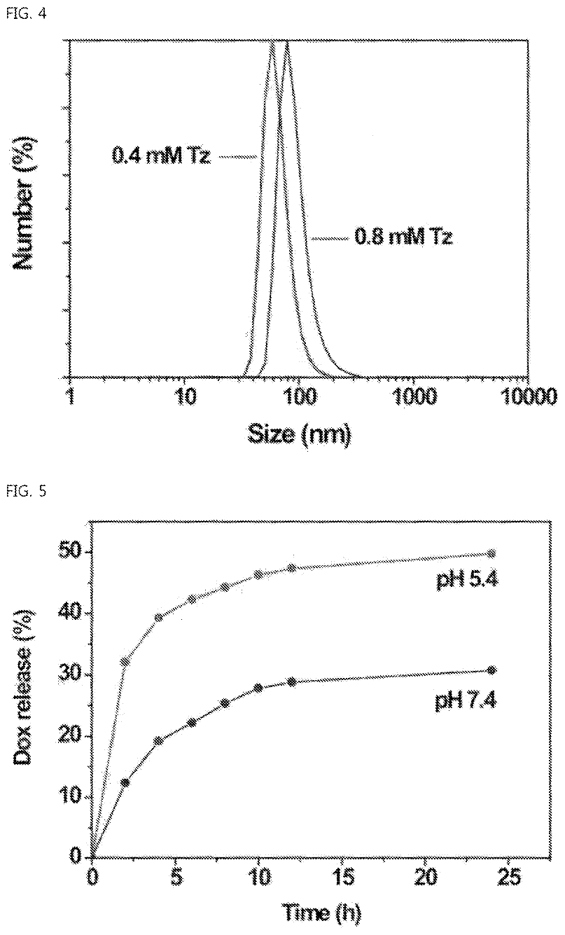

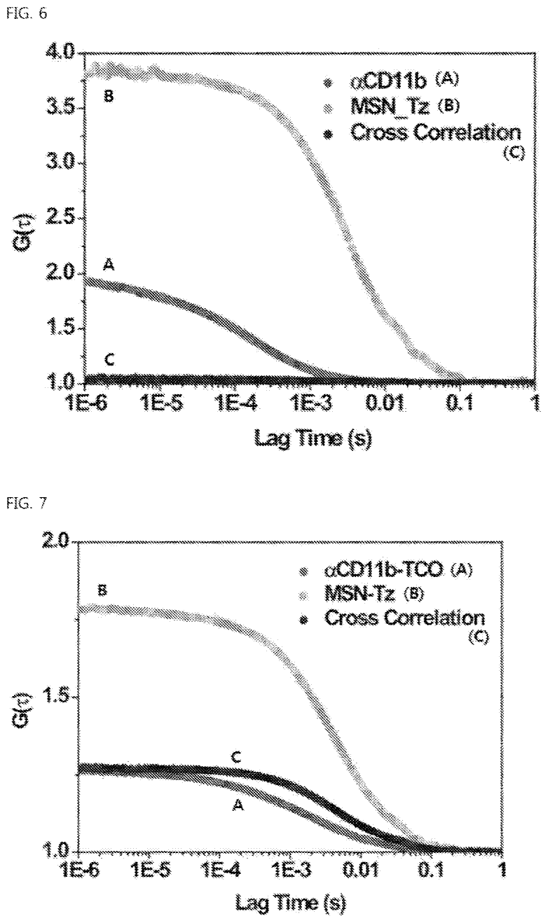

Referring to FIGS. 3 and 4, TEM images show that MSNs-Tz have spherical mesoporous nanostructures. The number average hydrodynamic diameter of MSNs-Tz determined by dynamic light scattering is about 66 nm. Since a large number of Tz molecules decorating the surface can increase the hydrodynamic diameter of MSN-Tz, the degree of Tz functionalization is optimized to keep the overall size below 100 nm. The colloidal stability of MSNs-Tz in biological media is investigated by fluorescence correlation spectroscopy (FCS). MSN-Tz incubated for 24 hours in 10% fetal bovine serum (FBS) cell media or PBS shows nearly identical FCS curves, demonstrating excellent colloidal stability of MSNs-Tz. According to UV-Vis absorption spectroscopy, the optimum number of Tz on the MSN surface to allow TCO molecules to react readily appears to be 77 molecules per MSN particle.

Fluorescent MSNs-Tz can be prepared by encapsulating rhodamine B isothiocyanate in the silica matrix of MSNs, and fluorescent MSNs-Tz show typical absorption and emission peaks at 561 nm and 587 nm, respectively.

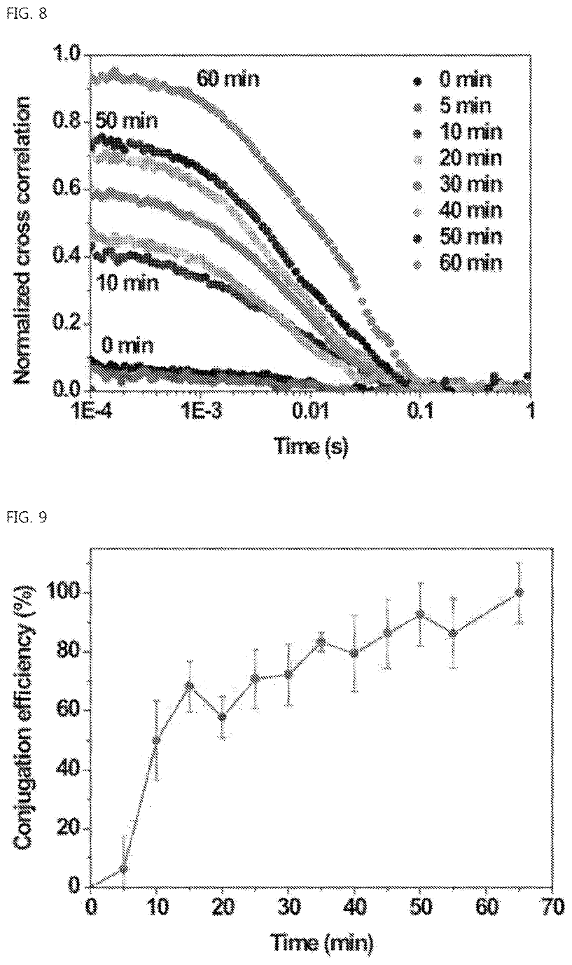

Referring to FIG. 5, doxorubicin is loaded in MSNs-Tz by physical adsorption, and the loaded doxorubicin is slowly and gradually released for 12 hours.

Anti-CD11b antibodies can be functionalized with TCO and fluorescent dye (Alexa Fluor 488). Each antibody can be functionalized with three TCO groups. UV-Vis absorption spectroscopy shows that a fast and selective click reaction occurs between Tz and TCO when anti-CD11b-TCO is incubated with excess amounts of Tz-Cy3 molecules. According to photoluminescence spectroscopy, the emission intensity of MSNs-Tz before the click reaction is low because the emission of rhodamine B within MSNs is partially quenched by Tz molecules on the surface. After the click reaction with anti-CD11b-TCO, the emission intensity increases by 1.7 fold because the resulting cyclic alkene does not absorb the emission of the rhodamine B dyes.

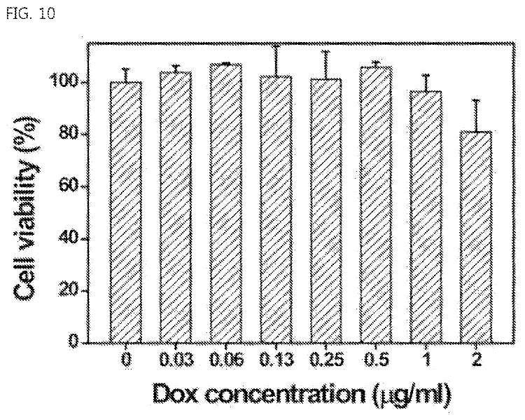

The kinetics of the click reaction between MSNs-Tz and anti-CD11b-TCO was investigated using dual color fluorescence cross-correlation spectroscopy (FCCS). FCCS can sensitively quantify the interactions between two spectrally distinct fluorophores and analyze the kinetics of addition reactions in real time, where chemical linkages are formed. To investigate the bioorthogonal reaction in the presence of serum proteins, fluorescent MSNs-Tz and anti-CD11b-TCO were reacted in 100% FBS at room temperature to simulate in vivo condition, and then FCCS measurements were performed every 10 min. Referring to FIGS. 6 and 7, control reactions with MSNs without Tz (Tz-omitting MSNs) and anti-CD11b without TCO (TCO-omitting) showed very weak cross-correlation because of the lack of specific reactions between MSNs and anti-CD11b. In contrast, strong cross-correlation was observed between MSNs-Tz and anti-CD11b-TCO. The antibody-MSN conjugates exhibit a diffusion coefficient (D=1.42 μm² s⁻¹) that is similar to MSNs-Tz (D=1.24 μm² s⁻¹), suggesting that no aggregation occurred.

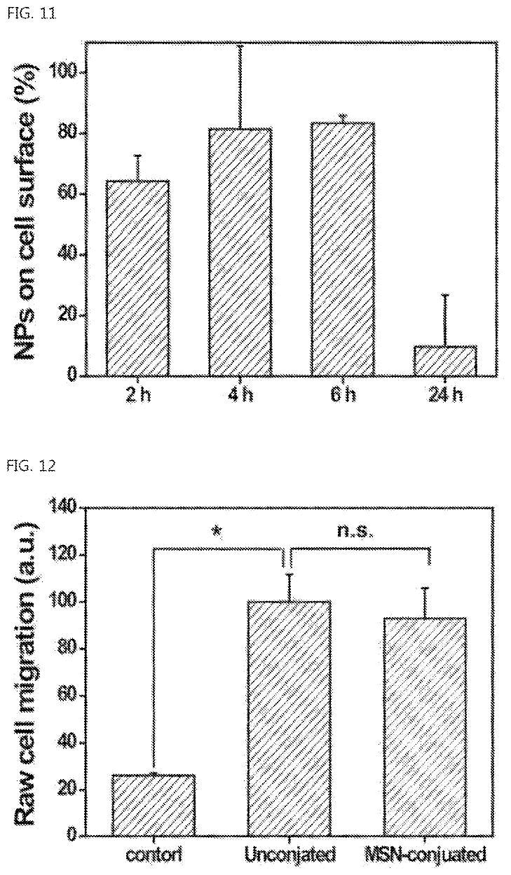

Referring to FIG. 8, the relative cross-correlation amplitude during the click reaction increases successively within 1 hour and subsequently remains constant.

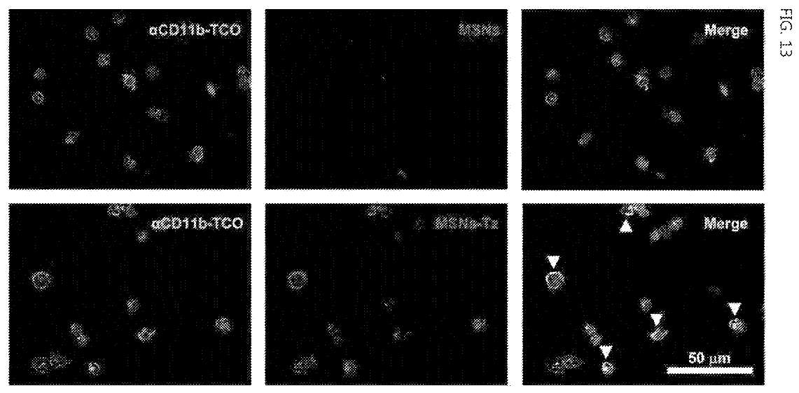

Referring to FIG. 9, the initial reaction rate of the click reaction increased with the addition of anti-CD11b-TCO, and the relative cross-correlation amplitude approached a plateau value after 40 min, indicating that the click reaction was completed within 40 min. These results indicate that anti-CD11b-TCO bound to the surface of the CD11b⁺ myeloid cell can rapidly bind to MSNs-Tz via bioorthogonal click reaction.

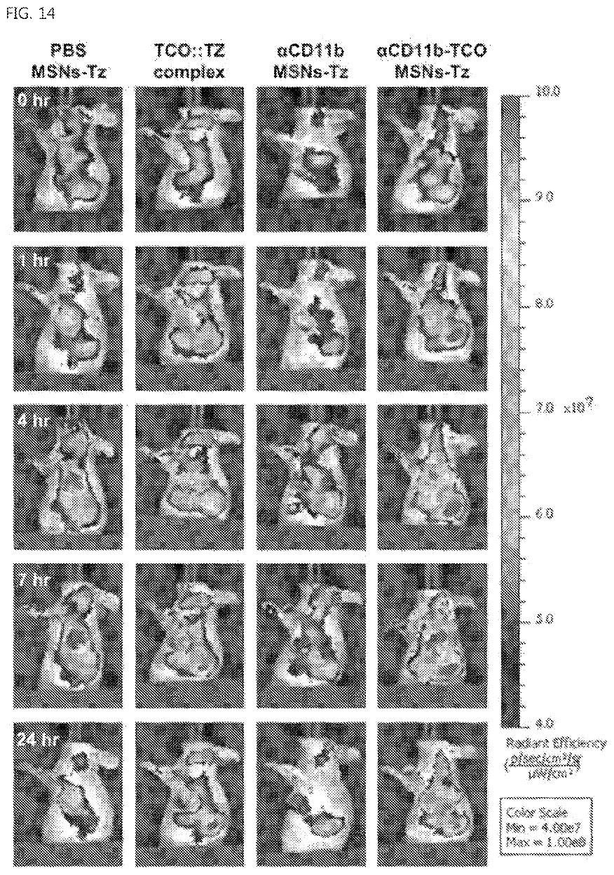

Because doxorubicin molecules can be toxic to normal cells, it was investigated whether CD11b⁺ myeloid cells tagged with doxorubicin-loaded MSNs-Tz remain viable and protected from doxorubicin molecules that may be released before reaching the tumor microenvironment. RAW 264.7 cells were tagged with anti-CD11b-TCO and subsequently conjugated with doxorubicin-loaded MSNs-Tz. Referring to FIG. 10, the doxorubicin-loaded MSNs-Tz have negligible toxicity compared to RAW cells having a doxorubicin concentration of 2 μg/ml. CD11b⁺ myeloid cells can carry therapeutic doses of doxorubicin without causing apoptosis.

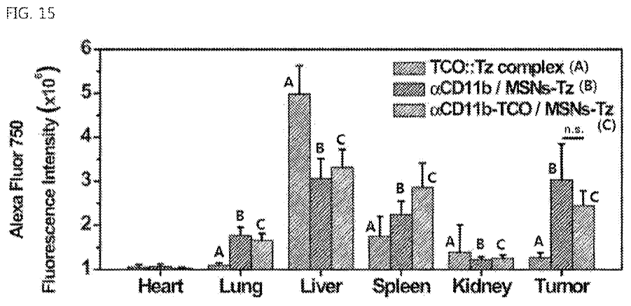

Referring to FIG. 11, Trypan blue quenching experiments show that about 80% of MSNs-Tz binds to RAW cells localized at the cell surface even after 6 hours incubation.

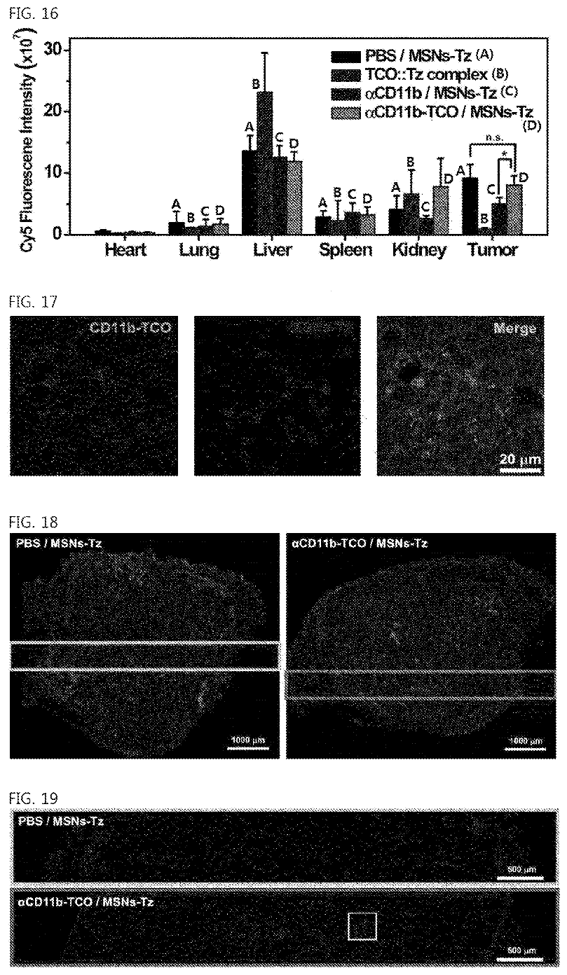

Migration of RAW cells conjugated with MSNs-Tz in response to chemoattractants derived from 4T1 tumor cells was assessed using an in vitro transwell co-culture system. Referring to FIG. 12, the conjugated cells exhibited migration capability similar to that of unmodified cells, showing that conjugation with MSNs-Tz does not affect cell migration.

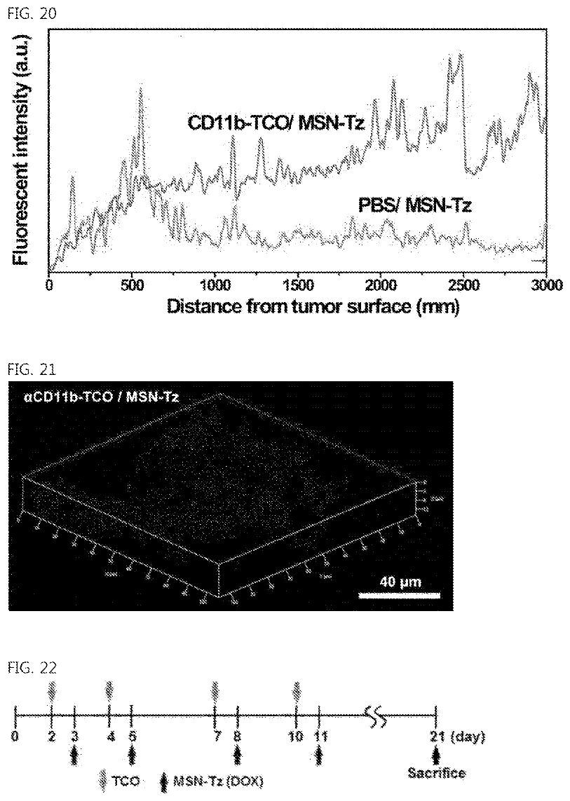

Referring to FIG. 13, confocal microscopy on endogenous bone marrow cells of 4T1 tumor-bearing mice was performed. Co-localization of fluorescent signals from anti-CD11b-TCO and MSNs-Tz was detected on the surface of bone marrow cells. In control experiments using antibodies without TCO or MSNs without Tz, only fluorescent signals were shown from the antibodies on the cell surface, and non-specific binding of MSNs-Tz to the bone marrow cells was hardly observed.

MSNs-Tz extravasate into the tumour interstitial space with a penetration depth of up to about 40 μm from the blood vessel. These confocal and intravital imaging data show that pretargeting with anti-CD11b antibodies and subsequent conjugation of MSNs-Tz by click reaction can selectively target circulating CD11b⁺ cells in vitro and in vivo. In addition, MSNs-Tz can be delivered to tumor sites via both the EPR effect and tagged CD11b⁺ cells.

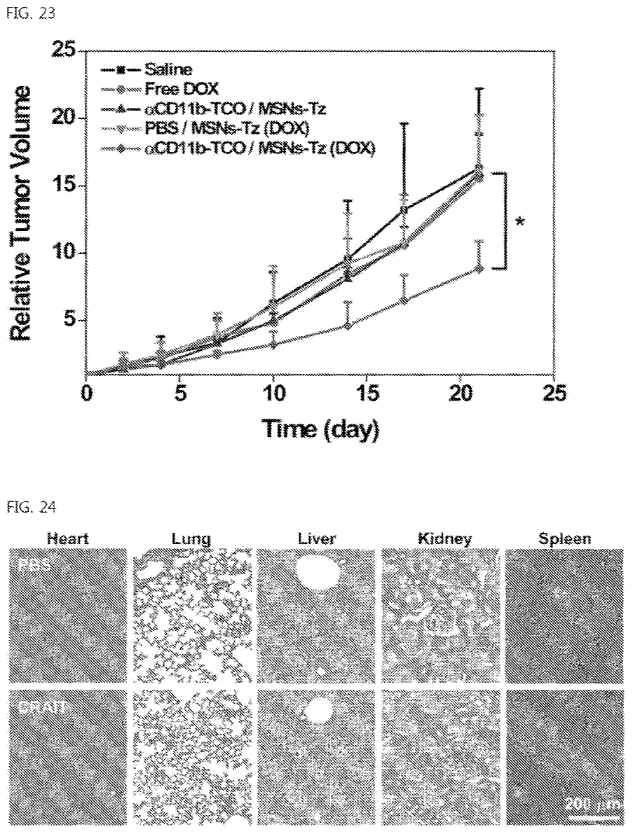

FIGS. 14 to 21 are views for explaining the delivery of MSNs-Tz to a 4T1 tumor in a living body using a drug delivery method according to an embodiment of the present invention. It was tested whether CD11b⁺ myeloid cells can transport doxorubicin-loaded MSNs-Tz into tumor in vivo. For more sensitive in vivo fluorescence imaging in the NIR range, the fluorophores of anti-CD11b-TCO and MSNs-Tz were modified with fluorescent dyes, Alexa Fluor 750 and Cy5, respectively.

(1) PBS/MSNs-Tz (nontargeted control group), (2) TCO::Tz complex (preconjugated group), (3) αCD11b/MSNs-Tz (TCO-omitting group), (4) αCD11b-TCO/MSN-Tz (embodiment group of the present invention) were injected via tail vein of mice bearing 4T1 tumors on the breast pad.

Referring to FIGS. 14 and 15, in vivo biodistribution of the anti-CD11b-TCO and MSNs-Tz were monitored by time-gated fluorescence imaging after injection. Biodistribution studies on anti-CD11b and anti-CD11b-TCO show similar accumulation in tumor, spleen and liver after 24 hours, confirming that TCO modification(functionalization) does not affect the targeting capability of the antibody.

Referring to FIG. 16, the MSNs-Tz of the embodiment group were accumulated in tumors at higher levels than the preconjugated group and the TCO-omitting group, but showed intratumoral accumulation similar to that of the nontargeted group. However, most of delivered MSNs-Tz by the EPR effect in the nontargeted group were located near tumor periphery, because tumor blood vessels were more abundant at the tumor-host interfaces than in internal regions.

In the case of the embodiment group, the negative effect associated with pre-targeting was reduced by the improved binding between CD11b antibodies and MSNs-Tz via click chemistry. Since labeled CD11b antibody scaffold has an average of three anchoring sites for subsequent click chemistry reaction, multiple MSNs-Tz can be attached to one antibody, thereby amplifying the loading of nanoparticles in tumors.

Referring to FIG. 17, fluorescence microscopy images obtained from tumor slices show that MSNs-Tz in the embodiment group are co-localized with anti-CD11b-TCO, whereas no co-localization is observed in the TCO-omitting group.

Referring to FIGS. 18 and 19, to evaluate tumor penetration and distribution of MSNs-Tz, tumors were excised after 24 hours and accumulation profile analysis was performed on the ex vivo histological specimens. From the representative tumor sections, MSNs-Tz in the embodiment group showed more uniform distribution and deeper penetration than the nontargeted group. Delivered MSNs-Tz in the embodiment group were found in both the peripheral and interior regions of tumors, while the nontargeted MSNs-Tz were predominantly localized at the periphery of the tumors.

Referring to FIG. 20, to quantify the penetration depth of MSNs-Tz, fluorescence intensities from tumor surface to central region were obtained. In the case of the nontargeted group, about 55% of overall fluorescence intensity was detected between tumor surface and 1 mm from tumor surface. Only 14% was observed between 2 mm and 3 mm from tumor surface, suggesting that their penetration is limited to tumor periphery. In contrast, about 35% of overall fluorescence intensity was observed between 2 mm and 3 mm from tumor surface in the embodiment group. Considering that tumor diameter is almost 5 mm, MSNs-Tz delivered in the embodiment group can penetrate up to about 2.5 mm from the tumor surface, corresponding to tumor interiors. At the tumor interior regions, MSNs-Tz delivered in the embodiment group exhibited about 5-fold higher fluorescence intensity than that of the nontargeted group. Deep tumor penetration of MSNs-Tz in the embodiment group can be attributed to the infiltration of labeled CD11b⁺ cells into tumour interiors, where hypoxic tumour cells secrete lysyl oxidase to recruit CD11b⁺ myeloid cells. This deep penetration implies that the drug delivery method according to the embodiments of the present invention can deliver drugs into hypoxic regions which are not accessible by conventional methods.

Referring to FIG. 21, three dimensional confocal images exhibited evenly dispersed MSNs-Tz in the volume.

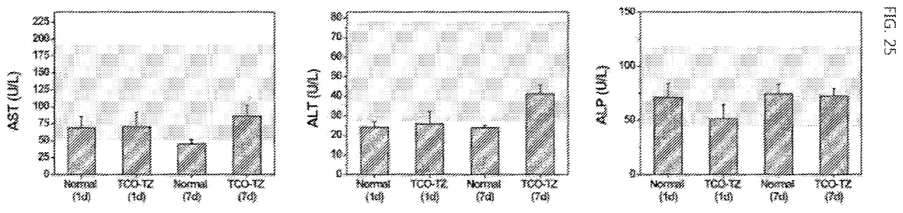

FIGS. 22 to 25 are views for explaining the therapeutic efficacy of MSNs-Tz improved by the drug delivery method according to an embodiment of the present invention. Mice bearing 4T1 tumors were treated with (1) PBS, (2) doxorubicin as free drug, (3) MSNs-Tz/anti-CD11b-TCO without doxorubicin, (4) nontargeted MSNs-Tz with doxorubicin (PBS/MSNs-Tz(DOX)), (5) doxorubicin-loaded MSNs-Tz(DOX)/anti-CD11b-TCO.

Referring to FIGS. 22 and 23, the amounts of doxorubicin in four groups are equivalent at 5 mg/kg, and all mice received a total of four treatments once every 3 days. Neither mice treated with free doxorubicin nor with MSNs-Tz/anti-CD11b-TCO without doxorubicin had an effect on tumor progression. The nontargeted MSNs-Tz with DOX, which are mainly delivered by the EPR effect, failed to inhibit tumor growth during the therapy because the nontargeted MSNs-Tz are heterogeneously distributed at tumor periphery with limited penetration. By contrast, treatment with the MSNs-Tz(DOX)/anti-CD11b-TCO induced about 2 fold reduction in tumor burden relative to free doxorubicin or untreated mice. These results demonstrate that the drug delivery method according to the embodiments of the present invention enhances the therapeutic efficacy of doxorubicin. CD11b⁺ cells, which act as drug carriers, deliver doxorubicin molecules deep inside the tumors, exposing large number of tumour cells to doxorubicin molecules.

Referring to FIGS. 24 and 25, the drug delivery method (CRAIT) according to the embodiments of the present invention does not exhibit toxicity in vivo. Healthy mice injected with PBS or CRAIT probes (anti-CD11b-TCO and MSN-Tz) showed no inflammatory sites or toxic damage in any of the major organs. In addition, serum measurements indicative of hepatic and renal toxicity fall within the range of healthy animals, indicating a lack of toxicity at all time points. Therefore, the drug delivery method according to the embodiments of the present invention improves the efficacy of doxorubicin without toxic side effects.

Although the embodiments of the present invention have been disclosed for illustrative purposes, those skilled in the art will appreciate that the present invention may be embodied in other specific ways without changing the technical spirit or essential features thereof. Therefore, the embodiments disclosed in the present invention are not restrictive but are illustrative. The scope of the present invention is given by the claims, rather than the specification, and also contains all modifications within the meaning and range equivalent to the claims.

### INDUSTRIAL APPLICABILITY

According to the embodiments of the present invention, it is possible to effectively deliver a drug to a target site in vivo, such as a tumor. Drug-loaded nanoparticles can penetrate deep into the tumor, improving the efficacy of tumor treatment. It does not require in vitro manipulation of cells and can be applied to various types of cells and nanovehicles.

1. A drug delivery system comprising:
a nanoparticle to which a first functional group is bound and drug is loaded;
an antibody to which a second functional group to react with the first functional group is bound; and
a carrier cell comprising an antigen protein to bind to the antibody.

2. The drug delivery system of claim 1, wherein the first functional group and the second functional group are bonded by a click reaction.

3. The drug delivery system of claim 1, wherein the first functional group comprises tetrazine and the second functional group comprises trans-cyclooctene.

4. The drug delivery system of claim 1, wherein the nanoparticle comprises polyethylene glycol disposed on the surface, and the first functional group binds to the polyethylene glycol.

5. The drug delivery system of claim 1, wherein the nanoparticle comprises mesoporous silica nanoparticle.

6. The drug delivery system of claim 1, wherein the antibody comprises an anti-CD11b antibody.

7. The drug delivery system of claim 1, wherein the carrier cell comprises a myeloid-derived suppressor cell.

8. The drug delivery system of claim 1, wherein the antigen protein comprises CD11b.

9. The drug delivery system of claim 1, wherein the nanoparticle penetrates a tumor in a living body by the carrier cell.

10. A drug delivery method comprising:
injecting a nanoparticle to which a first functional group is bound and drug is loaded, and an antibody to which a second functional group to react with the first functional group is bound into a living body; and
binding the antibody to an antigen protein of a carrier cell present in the living body, and binding the nanoparticle to the antibody by reaction of the first functional group and the second functional group.

11. The drug delivery method of claim 10, wherein the first functional group and the second functional group are bonded by a click reaction.

12. The drug delivery method of claim 10, wherein the first functional group comprises tetrazine and the second functional group comprises trans-cyclooctene.

13. The drug delivery method of claim 10, wherein the nanoparticle comprises polyethylene glycol disposed on the surface, and the first functional group binds to the polyethylene glycol.

14. The drug delivery method of claim 10, wherein the nanoparticle comprises mesoporous silica nanoparticle.

15. The drug delivery method of claim 10, wherein the antibody comprises an anti-CD11b antibody.

16. The drug delivery method of claim 10, wherein the carrier cell comprises a myeloid-derived suppressor cell.

17. The drug delivery method of claim 10, wherein the antigen protein comprises CD11b.

18. The drug delivery method of claim 10, wherein the nanoparticle penetrates a tumor in the living body by the carrier cell.
